(19) United States
(12) Patent Application Publication
(10) Pub. No.: US 2025/0276960 A1
(43) Pub. Date: Sep. 4, 2025

(54) CRYSTALLINE FORMS OF (R)-6-CHLORO-3-((1-(2-CYANO-7-METHYL-3-MORPHOLINOQUINOXALIN-5-YL)ETHYL)AMINO)PICOLINIC ACID

(71) Applicant: MIRATI THERAPEUTICS, INC., PRINCETON, NJ (US)

(72) Inventors: SAHAR ROSHANDEL, SAN DIEGO, CA (US); CATRINA JOU, SOUTH PLAINFIELD, NJ (US)

(73) Assignee: MIRATI THERAPEUTICS, INC., PRINCETON, NJ (US)

(21) Appl. No.: 19/047,751

(22) Filed: Feb. 7, 2025

Related U.S. Application Data
(60) Provisional application No. 63/551,369, filed on Feb. 8, 2024.

(51) Int. Cl. C07D 401/14; A61K 31/5377

(52) U.S. Cl. CPC C07D 401/14; A61K 31/5377

(57) ABSTRACT

The present invention relates to a crystalline form of (R)-6-chloro-3-((1-(2-cyano-7-methyl-3-morpholinoquinoxalin-5-yl) ethyl) amino) picolinic acid.

### CROSS REFERENCE TO RELATED APPLICATIONS

This application claims the benefit of U.S. Provisional Application No. 63/551,369, filed Feb. 8, 2024, the entire content of which is hereby incorporated herein by reference.

### FIELD OF THE INVENTION

This invention relates to a crystalline form of (R)-6-chloro-3- ((1-(2-cyano-7-methyl-3-morpholinoquinoxalin-5-yl) ethyl) amino) picolinic acid, processes of preparing the crystalline form, and pharmaceutical compositions including the crystalline form. The crystalline form thereof is useful in the treatment and/or prevention of diseases and/or conditions related to cell proliferation, such as cancer. In particular, the crystalline forms provide therapeutic benefits as inhibitors of phosphoinositide 3-kinase (PI3K).

### BACKGROUND OF THE INVENTION

The activity of cells can be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer, inflammatory disorders, metabolic disorders, vascular and neuronal diseases.

Kinases represent a class of important signaling molecules. Kinases can generally be classified into protein kinases and lipid kinases, and certain kinases exhibit dual specificities. Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates okay on tyrosine residues (e.g., erb2, PDGF receptor, EGF receptor, VEGF receptor, src, abl), serine/threonine kinases which predominantly phosphorylate substrates on serine and/or threonine residues (e.g., mTORC1, mTORC2, ATM, ATR, DNA-PK, Akt), and dual-specificity kinases which phosphorylate substrates on tyrosine, serine and/or threonine residues.

Lipid kinases are enzymes that catalyze the phosphorylation of lipids within cells. These enzymes, and the resulting phosphorylated lipids and lipid-derived biologically active organic molecules, play a role in many different physiological processes, including cell proliferation, migration, adhesion, and differentiation. A particular group of lipid kinases comprises membrane lipid kinases, i.e., kinases that catalyze the phosphorylation of lipids contained in or associated with cell membranes. Examples of such enzymes include phosphoinositide(s) kinases (such as PI3-kinases, PI4-Kinases), diacylglycerol kinases, and sphingosine kinases.

The phosphoinositide 3-kinases (PI3Ks) signaling pathway is one of the most highly mutated systems in human cancers. PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique and conserved family of intracellular lipid kinases that phosphorylate the 3′-OH group on phosphatidylinositols or phosphoinositides. The PI3K family comprises 15 kinases with distinct substrate specificities, expression patterns, and modes of regulation. The class I PI3Ks (pi 10a, pi 10b, pi 106, and pi 10g) are typically activated by tyrosine kinases or G-protein coupled receptors to generate PIP3, which engages downstream effectors such as those in the pathways of Akt/PDKI, mTOR, the Tec family kinases, and the Rho family GTPases. The class II and III PI3-Ks play a key role in intracellular trafficking through the synthesis of PI (3) P and PI (3,4) P2.

The PI3K isoforms have been implicated, for example, in a variety of human cancers and disorders. Mutations in the gene coding for PI3K isoforms or mutations which lead to upregulation of a PI3K isoform are believed to occur in many human cancers. Mutations in the gene coding for a PI3K isoform are point mutations clustered within several hotspots in helical and kinase domains. Because of the high rate of PI3K mutations, targeting of this pathway may provide valuable therapeutic opportunities.

Genetic alterations in genes in PI3K signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The alpha (α) isoform of PI3K has been implicated, for example, in a variety of human cancers. Angiogenesis has been shown to selectively require the alpha (α) isoform of PI3K in the control of endothelial cell migration. Mutations in the gene coding for PI3Kα or mutations which lead to upregulation of PI3Kα are believed to occur in many human cancers such as lung, stomach, endometrial, ovarian, bladder, breast, colon, brain, prostate, and skin cancers. Mutations in the gene coding for PI3Kα are point mutations clustered within several hotspots in helical and kinase domains, such as E542K, E545K, and H1047R. Many of these mutations have been shown to be oncogenic gain-of-function mutations. Because of the high rate of PI3Kα mutations, targeting of this pathway may provide valuable therapeutic opportunities. While other PI3K isoforms such as PI3Kδ or PI3Kγ are expressed primarily in hematopoietic cells, PI3Kα, along with PI3Kβ, is expressed constitutively.

Due to the central role of PI3Kα in regulating organismal glucose homeostasis, PI3K inhibition in patients often gives rise to hyperglycemia and/or hyperinsulinemia. High levels of circulating insulin could potentially be mitogenic and/or antiapoptotic for cancer cells and thus negate the antiproliferative effects of PI3K inhibitors.

In the setting of cancer with mutated PI3Kα, one way to overcome the problem of compensatory production of insulin and/or glucose upon systemic PI3Kα inhibition would be to develop inhibitors with enhanced selectivity for mutant PI3Kα over wild-type PI3Kα. This would create an increased window for drug dosing to selectively inhibit the pathologic signaling of mutant PI3Kα in the cancer cells without affecting the wild-type PI3Kα in the host tissues that control systemic metabolism, thus limiting toxicities and permitting higher doses and more complete inhibition of the drug target.

Existing PI3Kα inhibitors are nearly equipotent to wild-type and mutant PI3Kα. Mutant selective inhibitors have been elusive due to the PI3Kα mutations location far from the active site. As such, inhibitors which target a second, peripheral binding pocket near a known mutation (e.g., H1047R) may provide a route to selective PI3Kα inhibition. Thus, targeting a mutated, peripheral binding pocket of PI3Kα, may in turn provide a valuable therapeutic target for drug development.

As such, kinases, for example lipid kinases such as PI3Ks, are prime targets for drug development.

### SUMMARY OF THE INVENTION

In one aspect, this disclosure provide a crystalline form of (R)-6-chloro-3-((1-(2-cyano-7-methyl-3-morpholinoquinoxalin-5-yl) ethyl) amino) picolinic acid, shown below, and hereinafter Compound 1.

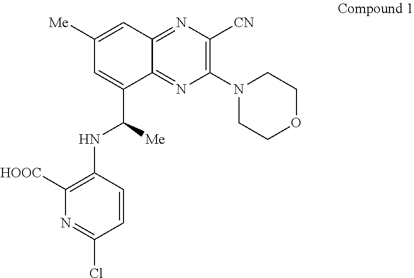 [SMILES: Cc1cc([C@@H](C)Nc2ccc(Cl)nc2C(=O)O)c2nc(N3CCOCC3)c(C#N)nc2c1]

This disclosure further provides processes of preparing a crystalline form of Compound 1. This disclosure further provides pharmaceutical compositions comprising a crystalline form of Compound 1 and a pharmaceutically acceptable carrier.

In another aspect, the present disclosure provides for a method of treating a disease or disorder associated with modulation of phosphoinositide 3-kinase (PI3K), comprising administering to a patient in need thereof a therapeutically effective amount of a crystalline form of Compound 1 as otherwise described herein or a pharmaceutical composition as otherwise described herein.

In another aspect, the present disclosure provides for a method of inhibiting phosphoinositide 3-kinase (PI3K), comprising administering to a patient in need thereof a therapeutically effective amount of a crystalline form of Compound 1 as otherwise described herein or a pharmaceutical composition as otherwise described herein.

In another aspect, the present disclosure provides for a method of treating cancer or a disorder, the method comprising administering to a patient in need thereof a therapeutically effective amount of a crystalline form of Compound 1 as otherwise described herein or a pharmaceutical composition as otherwise described herein.

### BRIEF DESCRIPTION OF THE FIGURES

FIG. 1A is a polarized light microscopy (PLM) image of an amorphous form of Compound 1.

FIG. 1B is a powder X-ray diffraction (PXRD) pattern of an amorphous form of Compound 1.

FIG. 2A is a polarized light microscopy (PLM) image of crystals of Compound 1.

FIG. 2B is a polarized light microscopy (PLM) image of crystals of Compound 1.

FIG. 3 is a powder X-ray diffraction (PXRD) pattern of a crystalline form of Compound 1.

FIG. 4 is a differential scanning calorimetric (DSC) thermogram and a thermal gravimetric analysis (TGA) overlay of a crystalline form of Compound 1.

FIG. 5 shows a proton nuclear magnetic resonance (¹H-NMR) spectrum of a crystalline form of Compound 1 and a proton nuclear magnetic resonance (¹H-NMR) spectrum of an amorphous form of Compound 1.

FIG. 6 is a high performance liquid chromatogram (HPLC) of a crystalline form of Compound 1.

FIG. 7A is a sorption isotherm of a crystalline form of Compound 1.

FIG. 7B is a dynamic vapor sorption trace of a crystalline form of Compound 1.

FIG. 8 is a powder X-ray diffraction (PXRD) pattern of a crystalline form of Compound 1 before and after dynamic vapor sorption.

FIG. 9A is a quantitative nuclear magnetic resonance (Q-NMR) spectrum of a crystalline form of Compound 1.

FIG. 9B is a quantitative nuclear magnetic resonance (Q-NMR) spectrum of a crystalline form of Compound 1.

### DETAILED DESCRIPTION OF THE INVENTION

As noted above, the invention provides a particular crystalline form of (R)-6-chloro-3-((1-(2-cyano-7-methyl-3-morpholinoquinoxalin-5-yl) ethyl) amino) picolinic acid.

The crystalline forms\as described herein may be characterized using a number of methods known to the person of ordinary skill in the art including thermal analysis (e.g., differential scanning calorimetry (DSC), thermal gravimetric analysis (TGA)), powder X-ray diffraction (PXRD), microscopy (e.g., scanning electron microscopy (SEM), polarized light microscopy), and spectroscopy (e.g., infrared, Raman, solid-sate nuclear magnetic resonance, proton nuclear magnetic resonance (¹HNMR)). The particle size and size distribution may be determined by conventional methods, such as laser light scattering technique. The purity of the crystalline forms provided herein may be determined by standard analytical methods, such as thin layer chromatography (TLC), gel electrophoresis, gas chromatography, high performance liquid chromatography (HPLC), mass spectroscopy (MS), and quantitative nuclear magnetic spectroscopy (Q-NMR).

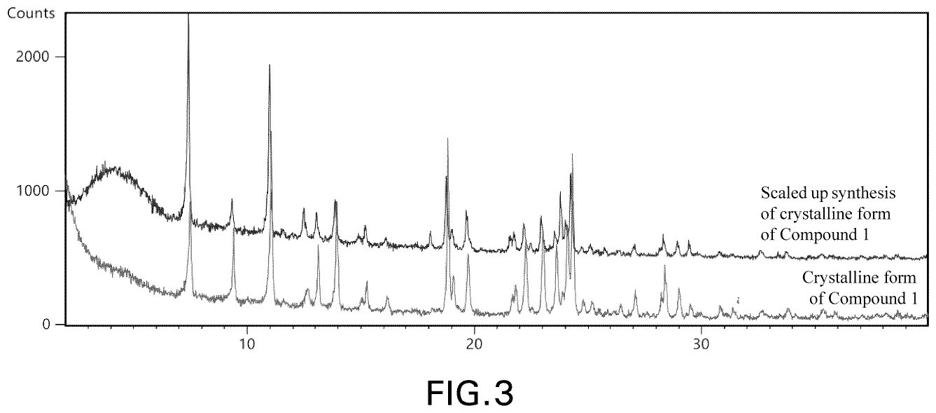

In one embodiment, this disclosure provides a crystalline form of (R)-6-chloro-3-((1-(2-cyano-7-methyl-3-morpholinoquinoxalin-5-yl) ethyl) amino) picolinic acid, i.e., Compound 1. In various embodiments, the crystalline form of Compound 1 has a powder X-ray diffraction (PXRD) pattern. In some embodiments, the crystalline form of Compound 1 has a PXRD pattern comprising a peak at a two-theta angle of 11.1°±0.2°. In some embodiments, the crystalline form of Compound 1 has a PXRD pattern comprising a peak at a two-theta angle of 18.8°±0.2°. In some embodiments, the crystalline form of Compounds 1 has a PXRD pattern comprising a peak at a two-theta angle of 24.3°±0.2°. In some embodiments, the crystalline form of Compound 1 has a PXRD pattern comprising a peak at a two-theta angle of 7.6°±0.2°. In some embodiments, the crystalline form of Compound 1 has a PXRD pattern comprising a peak at a two-theta angle of 13.1°±0.2°. In some embodiments, the crystalline form of Compound 1 has a PXRD pattern comprising a peak at a two-theta angle of 14.0°±0.2°. In some embodiments, the crystalline form of Compound 1 has a PXRD pattern comprising a peak at a two-theta angle of 19.7°±0.2°. In some embodiments, the crystalline form of Compound 1 has a PXRD pattern comprising a peak at a two-theta angle of 22.2°±0.2°. In some embodiments, the crystalline form of Compound 1 has a PXRD pattern comprising a peak at a two-theta angle of 23.0°±0.2°. In some embodiments, the crystalline form of Compound 1 has a PXRD pattern comprising a peak at a two-theta angle of 23.6°±0.2°. In some embodiments, the crystalline form of Compound 1 has a PXRD pattern comprising a peak at a two-theta angle of 24.1+±0.2°. In some embodiments, the crystalline form of Compound 1 has a PXRD pattern comprising peaks at a two-theta angle of 11.1°±0.2°, 18.8°±0.2°, and 24.3°±0.2°. In some embodiments, the crystalline form of Compound 1 has a PXRD pattern comprising peaks at a two-theta angle of 11.1°±0.2°, 18.8°±0.2°, 24.3°±0.2°, 7.6°±0.2°, 13.1°±0.2°, 14.0°±0.2°, 19.7°±0.2°, 22.2° ±0.2°, 23.0° ±0.2°, 23.6°±0.2°, and 24.1°±0.2°. For example, in some embodiments, the crystalline form of Compound 1 has a PXRD pattern substantially shown in FIG. 3.

In some embodiments as described herein, the crystalline form of Compound 1 has a unit cell of a=7.4±0.2 Å, b=18.8±0.2 Å, c=30.4±0.2 Å, α=90°, β=90°, and γ=90°. As would be understood by the person of ordinary skill in the art, a, b, and c define the cell edges and α, β, and γ define the angles between them with a being the angle between b and c, β is the angle between a and c, and γ is the angel between a and b.

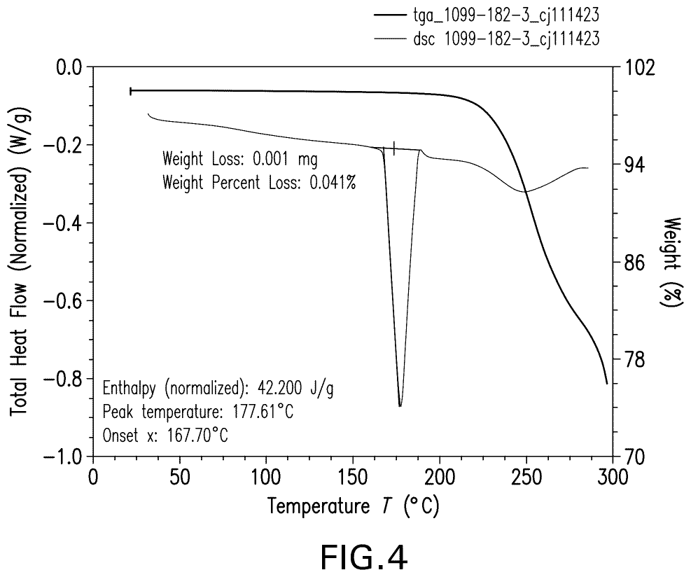

In various embodiments, the crystalline form of Compound 1 has a differential scanning calorimetric (DCS) thermogram (e.g., comprising endothermic peaks). In some embodiments, the crystalline form of Compound 1 has an endothermic DSC peak of about 177° C., e.g., within about ±2% of 177° C. In some embodiments, the crystalline form of Compound 1 has an endothermic DSC peak temperature within ±1% of 177° C. or within ±0.5% of 177° C. For example, in some embodiments, the crystalline form of Compound 1 has a DSC thermogram substantially shown in FIG. 4.

In some embodiments as described herein, the crystalline form of Compound 1 is anhydrous. In some embodiments as described herein, the crystalline form of Compound 1 is non-hygroscopic.

In some embodiments as described herein, the crystalline form of Compound 1 has a thermal gravimetric analysis (TGA) plot comprising a mass loss of about 0.041% when heated from about 25° C. to about 100° C. For example, in some embodiments, the crystalline form of Compound 1 has a TGA plot substantially shown in FIG. 4. In some embodiments as described herein, the crystalline form of Compound 1 has substantially no thermal degradation at temperatures below about 210° C. In some embodiments as described herein, the crystalline form of Compound 1 has some thermal degradation at temperatures above about 210° C.

In some embodiments as described herein, the crystalline form of Compound 1 has a purity of at least 97% by weight of (R)-6-chloro-3-((1-(2-cyano-7-methyl-3-morpholinoquinoxalin-5-yl) ethyl) amino) picolinic acid. In some embodiments, the crystalline form of Compound 1 has a purity of at least 98% by weight of (R)-6-chloro-3-((1-(2-cyano-7-methyl-3-morpholinoquinoxalin-5-yl) ethyl) amino) picolinic acid.

### Processes of Preparing a Crystalline Form of Compound 1

In another aspect, this disclosure provides processes of preparing the crystalline form of Compound 1, as described herein. The crystalline form of Compound 1 can be made by a variety of methods and is not particularly limited. For example, the crystalline form as described herein may be prepared by slurry methods.

To prepare the crystalline form with the slurry method, amorphous solids of Compound 1 are suspended and stirred in a solvent at a temperature (e.g., room temperature) to provide solids. In various embodiments, the slurry method can be conducted at a variety of temperatures and with a variety of solvents. For example, in some embodiments, the solvent may be selected from methanol (MeOH), ethanol (EtOH), isopropyl alcohol (IPA), isopropyl acetate (IPAc), ethyl acetate (EtOAc), n-propyl alcohol (nPA), acetonitrile (ACN), methyl ethyl ketone (MEK), methyl isobutyl ketone (MIBK), methyl tert-butyl ether (MTBE), tetrahydrofuran (THF), 1,4-dioxane, 2-methyl tetrahydrofuran (2-MeTHF), heptane, toluene, water, acetone, dichloromethane (DCM), dimethylformamide (DMF), or dimethyl sulfoxide (DMSO). In some embodiments, the slurry method is conducted at room temperature.

### Pharmaceutical Compositions

In another aspect, this disclosure provides pharmaceutical comprising the crystalline form of Compound 1, as described herein, and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds. In certain embodiments, the composition may include one or more antibiotic compounds.

Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. Pharmaceutically acceptable salts may also be prepared as alkali metal salts or alkaline earth metal salts, such as sodium salts, potassium salts or calcium salts. Examples of such alkali metal salts or alkaline earth metal salts include, but are not limited to basic addition salts formed with inorganic bases (for example, hydroxides, carbonates, or bicarbonates). The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The crystalline form of Compound 1 is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 500 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

### Methods of Treatment

In another aspect, the present disclosure provides a method of treating a disease or disorder associated with modulation of phosphoinositide 3-kinase (PI3K), comprising administering to a patient in need thereof a therapeutically effective amount of the crystalline form of Compound 1 as otherwise described herein or a pharmaceutical composition as otherwise described herein. For example, in some embodiments, the PI3K is PI3Kα. In some embodiments as described herein, the PI3K is associated with the disease or disorder has a H1047R mutation.

In some embodiments as described herein, the disease or disorder is cancer. For example, in various embodiments, the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

In some other embodiments as described herein, the disease or disorder is CLOVES syndrome (congenital lipomatous overgrowth, vascular malformations, epidermal naevi, scoliosis/skeletal and spinal syndrome), or PIK3Cα-related overgrowth syndrome (PROS).

In another aspect, the present disclosure provides a method of inhibiting phosphoinositide 3-kinase (PI3K), comprising administering to a patient in need thereof a therapeutically effective amount of a crystalline form of Compound 1 as otherwise described herein or a pharmaceutical composition as other described herein.

In another aspect, the present disclosure provides a method of treating cancer or a disorder, the method comprising administering to a patient in need thereof a therapeutically effective amount of a crystalline form of Compound 1 as otherwise described herein or a pharmaceutical composition as otherwise described herein. In various embodiments as described herein, the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

In another aspect, the present disclosure provides a method of treating cancer comprising administering a crystalline form of Compound 1 as otherwise described herein or a pharmaceutical composition as otherwise described herein and a KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

In another aspect, the present disclosure provides a method treating cancer comprising administering a crystalline form of Compound 1 as otherwise described herein or a pharmaceutical composition as otherwise described herein and a mutant selective KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

In another aspect, the present disclosure provides a crystalline form as otherwise described herein or a pharmaceutical composition as otherwise described herein for use in the treatment of cancer in combination with a KRAS inhibitor, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

### EXAMPLES

The following Examples are intended to illustrate further certain embodiments of the invention and are not intended to limit the scope of the invention.

### Example 1: Preparation of Compound 1

Schemes A, B and C and the procedures are described in detail below.

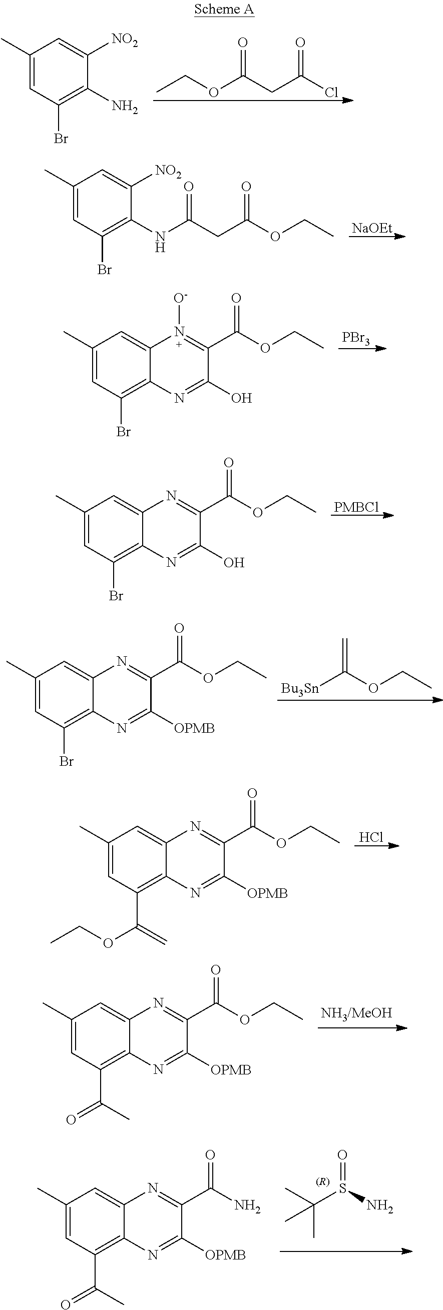

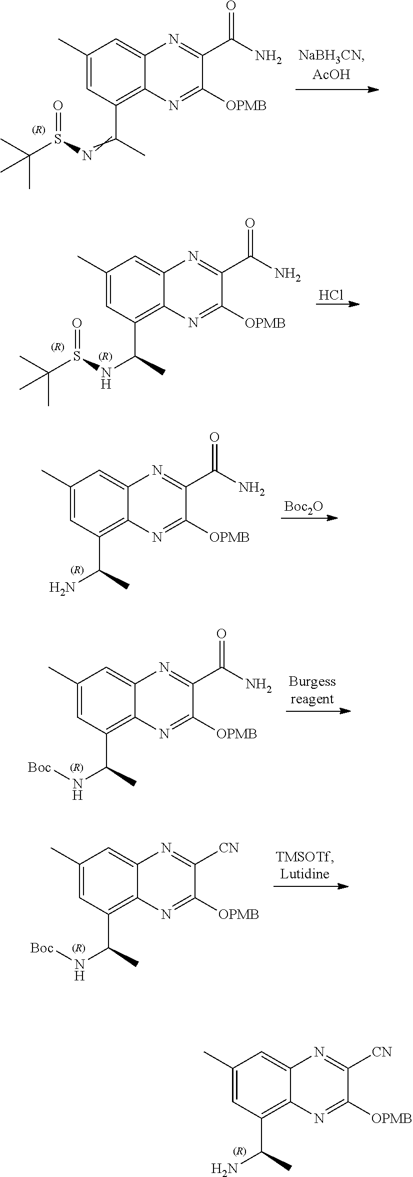

Step A: To a solution of 2-bromo-4-methyl-6-nitroaniline (540 g, 2.34 mol, 1.00 eq.) in toluene (650 mL) was added ethyl 3-chloro-3-oxopropanoate (422 g, 2.80 mol, 353 mL, 1.20 eq.) at 0° C., then the reaction was stirred at 110° C. for 1 hour. After completion of the reaction, the mixture was cooled to 25° C. and poured into petroleum ether (3.00 L). The mixture was filtered and the filtered cake was collected to give ethyl 3-((2-bromo-4-methyl-6-nitrophenyl) amino)-3-oxopropanoate (790 g, 2.29 mol, 97.9% yield) as a yellow solid. LCMS [M+1]⁺⁼344.9.

¹H NMR (400 MHZ, CDCl₃) δ32 9.70 (br s, 1H), 7.70 (d, J=2.0 Hz, 2H), 4.30 (q, J=7.2 Hz, 2H), 3.51 (s, 2H), 2.41 (s, 3H), 1.35 (t, J=7.2 Hz, 3H).

Step B: To a solution of sodium ethoxide (734 mL, 2.17 M, 2.20 eq.) in ethanol (500 mL) and tetrahydrofuran (1.50 L) was added a solution of ethyl 3-((2-bromo-4-methyl-6-nitrophenyl) amino)-3-oxopropanoate (250 g, 724 mmol, 1.00 eq.) in tetrahydrofuran (1.00 L) dropwise at −30° C. Then the reaction was stirred at −30° C. for 15 minutes. After completion of the reaction, the mixture was poured into ice water (5.00 L) slowly at 0° C. Then the pH of the mixture was adjusted with hydrogen chloride (4 M in water) to pH ˜4. The resulting suspension was filtered and the filtered cake was collected to give a crude product. The crude product was triturated with acetonitrile (750 mL) at room temperature for 30 minutes. Then the mixture was filtered, and the filter cake was collected, dried over under reduced pressure to give 5-bromo-2-(ethoxycarbonyl)-3-hydroxy-7-methylquinoxaline 1-oxide (190 g, 542 mmol, 74.9% yield, 93.4% purity) as a yellow solid. LCMS [M+3]⁺=328.9.

¹H NMR (400 MHZ, CDC13) δ=9.67 (br s, 1H), 8.09 (s, 1H), 7.71 (d, J=1.2 Hz, 1H), 4.52 (q, J=7.2 Hz, 2H), 2.46 (s, 3H), 1.43 (t, J=6.8 Hz, 3H).

Step C: To a solution of 5-bromo-2-(ethoxycarbonyl)-3-hydroxy-7-methylquinoxaline 1-oxide (200 g, 581 mmol, 1.00 eq.) in DMF (2.50 L) was added phosphorus tribromide (314 g, 1.16mol, 110 mL, 2.00 eq.) dropwise at 0° C. Then the reaction was stirred at 60° C. for 2 hours. The mixture was then cooled to 25° C. and poured into ice water (4.00 L). The resulting mixture was filtered and the filter cake was collected. The filter cake was triturated with acetonitrile (500 mL) at 15° C. for 30 minutes. Then the mixture was filtered, and the filter cake was collected, dried over under reduced pressure to give ethyl 5-bromo-3-hydroxy-7-methylquinoxaline-2-carboxylate (150 g, 465 mmol, 80.1% yield, 96.5% purity) as a yellow solid. LCMS [M+1]⁺=310.9.

¹H NMR (400 MHZ, CDCl3) δ=9.54 (br s, 1H), 7.71 (s, 1H), 7.66 (s, 1H), 4.51 (q, J=7.2 Hz, 2H), 2.45 (s, 3H), 1.44 (t, J=7.2 Hz, 3H).

Step D: To a solution of ethyl 5-bromo-3-hydroxy-7-methylquinoxaline-2-carboxylate (300 g, 964 mmol, 1.00 eq.) and potassium carbonate (400 g, 2.89 mol, 3.000 eq.) in DMF (2.40 L) was added PMBCI (151 g, 964 mmol, 131 mL, 1.00 eq.), and the reaction was stirred at 50° C. for 8 hours. The mixture was then cooled to 25° C. and then poured into ice water (4.00 L). Then the mixture was filtered, and the filter cake was collected. The filter cake was triturated with acetonitrile (800 mL) at 20° C. for 20 minutes, then filtered and the filter cake was again collected, and dried over under reduced pressure to give ethyl 5-bromo-3- ((4-methoxybenzyl) oxy)-7-methylquinoxaline-2-carboxylate (380 g, 819 mmol, 85.0% yield, 93.0% purity) as a yellow solid. LCMS [M+23]⁺=453.1.

¹H NMR (400 MHZ, CDCl3) δ=7.86 (s, 1H), 7.79 (s, 1H), 7.56 (d, J=8.4 Hz, 2H), 6.89 (d, J=8.4 Hz, 2H), 5.61 (s, 2H), 4.49 (q, J=6.8 Hz, 2H), 3.78 (s, 3H), 2.49 (s, 3H), 1.41 (t, J=7.2 Hz, 3H).

Step E: A mixture of ethyl 5-bromo-3-((4-methoxybenzyl) oxy)-7-methylquinoxaline-2-carboxylate (1.00 kg, 2.16 mol, 1.00 eq.), tributyl (1-ethoxyvinyl) tin (1.00 kg, 2.77 mol, 935 mL, 1.28 eq.), Pd₂(dba)₃ (98.7 g, 108 mmol, 0.050 eq.) and XPhos (51.4 g, 108 mmol, 0.050 eq.) in toluene (16.0 L) was degassed and purged with nitrogen 3 times. The reaction was then stirred at 100° C. for 5 hours under a nitrogen atmosphere. After completion of the reaction, the mixture was cooled to 25° C. and then the solution of ethyl 5-(1-ethoxyvinyl)-3-((4-methoxybenzyl) oxy)-7-methylquinoxaline-2-carboxylate (911 g, crude) in toluene (16.0 L) was obtained as a black liquid, which was used directly in the next step. LCMS [M+1]⁺=423.2.

Step F: A solution of ethyl 5-(1-ethoxyvinyl)-3-((4-methoxybenzyl) oxy)-7-methylquinoxaline-2-carboxylate (911 g, crude) in toluene (16.0 L) was cooled to 20° C. and tetrahydrofuran (14.0 L) was added. To this mixture was added hydrogen chloride (1.94 L, 1.00 M, 0.900 eq.) dropwise at 0° C., and the mixture was stirred at 0° C. for 30 minutes. After this time, the pH of the mixture was adjusted with sodium hydrogen carbonate (saturated in water) to pH ˜7. To this mixture was added potassium fluoride (4.31 L, 4.00 M in water). After 1 hour, the mixture was diluted with water (6.00 L) and ethyl acetate (6.00 L). The mixture was then filtered, and the filtrate was extracted with ethyl acetate (5.00 L×2). The combined organic layers were dried over anhydrous sodium sulfate, filtered, and concentrated under reduced pressure to give a residue. The residue was triturated with (dichloromethane: ethyl acetate =1:2, 500 mL) and then further triturated with (petroleum ether: ethyl acetate =3:1, 500 mL) at 20° C. for 30 minutes. The mixture was then filtered and the filter cake was collected, and dried under reduced pressure to give ethyl 5-acetyl-3-((4-methoxybenzyl) oxy)-7-methylquinoxaline-2-carboxylate (640 g, 1.51 mol, 70.0% yield) as a yellow solid. LCMS [M+23]⁺=417.1.

¹H NMR (400 MHZ, CDCl₃) δ=8.04 (d, J=0.8 Hz, 1H), 7.99 (d, J=2.0 Hz, 1H), 7.42 (d, J=8.8 Hz, 2H), 6.92 (d, J =8.4 Hz, 2H), 5.53 (s, 2H), 4.51 (q, J =6.8 Hz, 2H), 3.82 (s, 3H), 2.93 (s, 3H), 2.57 (s, 3H), 1.41 (t, J=7.2 Hz, 3H).

Step G: Ammonia (130 g, 7.62 mol, 14.6 eq.) was bubbled into a solvent of methanol (1.00 L) at 15° C. for 1 hour. Then the above solution was added to a mixture of ethyl 5-acetyl-3-((4-methoxybenzyl) oxy)-7-methylquinoxaline-2-carboxylate (206 g, 522 mmol, 1.00 eq.) in methanol (2.00 L). The reaction was stirred at 25° C. for 16 hours., then concentrated at 30° C. under reduced pressure to give 5-acetyl-3-((4-methoxybenzyl) oxy)-7-methylquinoxaline-2-carboxamide (210 g, 466 mmol, 89.1% yield, 81.0% purity) as a yellow solid. LCMS [M+1]⁺=366.3.

Step H: To a mixture of 5-acetyl-3-((4-methoxybenzyl) oxy)-7-methylquinoxaline-2-carboxamide (89.0 g, 244 mmol, 1.00 eq.) and (R)-2-methylpropane-2-sulfinamide (148 g, 1.22mol, 5.00 eq.) in 2-methyltetrahydrofuran (3.00 L) was added titanium (IV) ethoxide (278 g, 1.22mol, 253 mL, 5.00 eq.), and the reaction was stirred at 70° C. for 24 hours. The mixture was then cooled to 25° C., diluted with ethyl acetate (2.00 L) and quenched using sodium hydrogen carbonate (saturated in water, 1.50 L) at 0° C. The mixture was then filtered and the filtrate was washed with water (1.00 L × 2), followed by brine (500 mL ×1), dried over anhydrous sodium sulfate, filtered, and concentrated under reduced pressure to give a residue. The residue was triturated with ethyl acetate (100 mL) at 20° C. for 1 hour. Then the mixture was filtered and the filter cake was collected, dried over under reduced pressure to give (R)-5-(1-((tert-butylsulfinyl) imino) ethyl)-3-((4-methoxybenzyl) oxy)-7-methylquinoxaline-2-carboxamide (101g, 174 mmol, 71.4% yield, 80.7% purity) as a yellow solid. LCMS [M+1]⁺=469.2.

¹H NMR (400 MHz, CDCl₃) δ=7.98 (s, 1H), 7.73 (s, 1H), 7.59 (br s, 1H), 7.45-7.42 (m, 2H), 6.91 (br d, J=6.8 Hz, 2H), 6.23 (br s, 1H), 5.55 (s, 2H), 3.81 (s, 3H), 2.94 (d, J=1.6 Hz, 3H), 2.57 (s, 3H), 1.23 (s, 9H).

Step I: To a solution of (R)-5-(1-((tert-butylsulfinyl) imino) ethyl)-3-((4-methoxybenzyl) oxy)-7-methylquinoxaline-2-carboxamide (155 g, 257 mmol, 1.00 eq.) in dichloromethane (1.50 L) and methanol (1.50 L) were added acetic acid (77.1 g, 1.28 mol, 73.5 mL, 5.00 eq.) and sodium cyanoborohydride (64.5 g, 1.03 mol, 4.00 eq.) at 0° C. The reaction was then stirred at 20° C. for 12 hours. After this time, the mixture was washed with water (1.00 L) and the organic phase was treated with sodium hydrogen carbonate (saturated in water, 800 mL) slowly at 0° C. The resultant mixture was washed with water (1.00 L) and brine (500 mL), dried over anhydrous sodium sulfate, filtered, and concentrated under reduced pressure to give 5-((R)-1-(((R)-tert-butylsulfinyl) amino) ethyl)-3-((4-methoxybenzyl) oxy)-7-methylquinoxaline-2-carboxamide (155g, crude) as a yellow solid. LCMS [M+1]⁺=471.2.

Step J: To a solution of 5-((R)-1-(((R)-tert-butylsulfinyl) amino) ethyl)-3-((4-methoxybenzyl) oxy)-7-methylquinoxaline-2-carboxamide (190 g, 299 mmol, 1.00 eq.) in THF (2.00 L) was added hydrogen chloride (380 mL, 1.00 M in water, 1.27 eq.) dropwise at 0° C., and the mixture was stirred at 15° C. for 7 hours. After the reaction was completed the solution of (R)-5-(1-aminoethyl)-3-((4-methoxybenzyl) oxy)-7-methylquinoxaline-2-carboxamide (160 g, crude) in THF (2.00 L) was obtained as yellow liquid, which was used directly in the next step. LCMS [M+1]⁺=367.1.

Step K: To a solution of ((R)-5-(1-aminoethyl)-3-((4-methoxybenzyl) oxy)-7-methylquinoxaline-2-carboxamide (160 g, 288 mmol, 1.00 eq.) in THF (2.00 L) was added sodium hydroxide (34.6g, 865 mmol, 3.00 eq.) in water (1.00 L) and Boc₂O (75.5 g, 346 mmol, 79.4 mL, 1.20 eq.), and the reaction was stirred at 50° C. for 1 hour. After this time, the mixture was diluted with ethyl acetate (1.00 L), washed with brine (500 mL×2), dried over anhydrous sodium sulfate, filtered, and concentrated under reduced pressure to give tert-butyl (R)-(1-(2-carbamoyl-3-((4-methoxybenzyl) oxy)-7-methylquinoxalin-5-yl) ethyl) carbamate (160 g, crude) as a yellow solid. LCMS [M+1]⁺=467.3.

Step L: To a solution of tert-butyl (R)-(1-(2-carbamoyl-3-((4-methoxybenzyl) oxy)-7-methylquinoxalin-5-yl) ethyl) carbamate (215 g, 277 mmol, 1.00 eq.) in THF (3.00 L) was added Burgess reagent (198 g, 830 mmol, 3.00 eq.) at 0° C. The reaction was stirred at 0° C. for 1 hour, then diluted with ethyl acetate (1.00 L), washed with water (500 mL) and brine (500 mL×2). The organic phase was dried over anhydrous sodium sulfate, filtered, and concentrated under reduced pressure to give a residue. The residue was triturated with acetonitrile (180 mL) at 20° C. for 30 minutes, then filtered and the filter cake was collected, and dried over reduced pressure to give tert-butyl (R)-(1-(2-cyano-3-((4-methoxybenzyl) oxy)-7-methylquinoxalin-5-yl) ethyl) carbamate (125 g, 234 mmol, 84.7% yield, 84% purity) as a yellow solid. LCMS [M+23]⁺=471.3.

¹H NMR (400 MHZ, CDCl₃) δ=7.73 (d, J=0.8 Hz, 1H), 7.60 (d, J=1.6 Hz, 1H), 7.52-7.47 (m, 2H), 6.97-6.91 (m, 2H), 5.69-5.40 (m, 4H), 3.82 (s, 3H), 2.54 (s, 3H), 1.53 (d, J=6.8 Hz, 3H), 1.44 (br s, 9H).

Step M: To a solution of tert-butyl (R)-(1-(2-cyano-3-((4-methoxybenzyl) oxy)-7-methylquinoxalin-5-yl) ethyl) carbamate (300 g, 562 mmol, 1.00 eq.) in dichloromethane (3.00 L) was added 2,6-dimethylpyridine (482 g, 4.49 mol, 524 mL, 8.00 eq.) and trimethylsilyl trifluoromethanesulfonate (500 g, 2.25 mol, 406 mL, 4.00 eq.), then the mixture was stirred at 40° C. for 1 hour, followed by dilution with water (2.50 L) at 0°° C. The resulting mixture was washed with brine (500 mL×2) and then citric acid (1 N in water, 1000 mL×4). The combined organic layers were dried over anhydrous sodium sulfate, filtered, and concentrated under reduced pressure to give a residue. The residue was triturated with acetonitrile (450 mL) at 20° C. for 10 minutes. Then the mixture was filtered, and the filter cake was collected, and dried under reduced pressure to give the crude product. The crude product was purified by reversed phase flash (column: Sfar C18 1800 g D Duo 30 um; mobile phase: phase A: 0.225% formic acid in water, phase B: acetonitrile; gradient: 35%-65% phase B) to give (R)-5-(1-aminoethyl)-3-((4-methoxybenzyl) oxy)-7-methylquinoxaline-2-carbonitrile (195 g, 535 mmol, 95.2% yield, 95.6% purity) as a yellow solid. LCMS [M+1] +=349.1.

¹H NMR (400 MHZ, CDCl₃) δ=7.71 (s, 2H), 7.47 (d, J=8.4 Hz, 2H), 6.92 (d, J=8.4 Hz, 2H), 5.55 (s, 2H), 5.01 (q, J=6.8 Hz, 1H), 3.81 (s, 3H), 2.69 (br s, 2H), 2.54 (s, 3H), 1.55 (d, J=6.8 Hz, 3H).

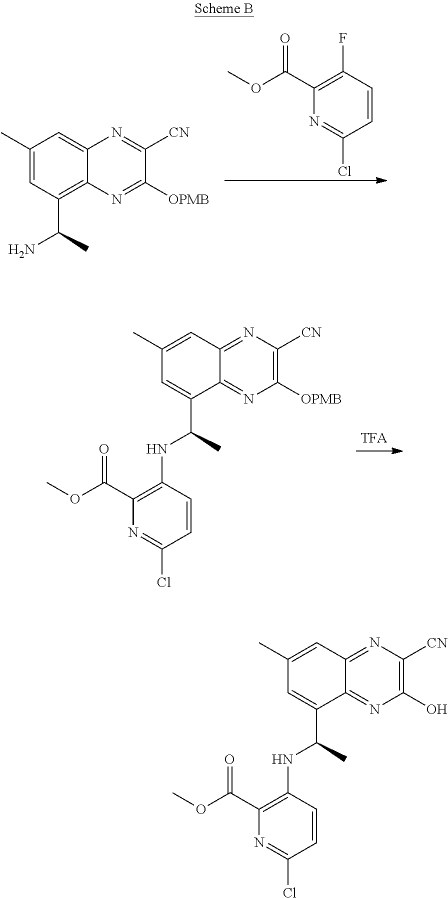

Step N: A mixture of (R)-5-(1-aminoethyl)-3-((4-methoxybenzyl) oxy)-7-methylquinoxaline-2-carbonitrile (2.67 g, 7.66 mmol, 1.00 eq.), methyl 6-chloro-3-fluoropicolinate (2.03 g, 10.7mmol, 1.40 eq.), diisopropylethylamine (4.95 g, 38.3 mmol, 6.67 mL, 5.00 eq.) in N,N-dimethylformamide (30.0 mL) was degassed and purged with dinitrogen for 3 times, and then the mixture was stirred at 100° C. for 12 hours under dinitrogen atmosphere. After completion, the mixture was cooled to 20° C. The mixture was poured into water aqueous solution (10.0 mL) and extracted with ethyl acetate (30.0 mL×3). The combined organic layer was washed with brine (10.0 mL), dried over anhydrous sodium sulfate, then the mixture was filtered and the filtrate was concentrated. The residue was purified by column chromatography (SiO2, petroleum ether/ethyl acetate=1/0 to 3/1) to give methyl (R)-6-chloro-3-((1-(2-cyano-3-((4-methoxybenzyl) oxy)-7-methylquinoxalin-5-yl) ethyl) amino) picolinate (2.30 g, 4.00 mmol, 52.2% yield, 90% purity) as a yellow solid. LCMS [M+23] ⁺=540.2.

¹H NMR (400 MHZ, DMSO-d6) δ=8.40 (d, J=7.20 Hz, 1H), 7.88-7.76 (m, 2H), 7.52 (d, J=8.40 Hz, 2H), 7.28 (d, J=9.20 Hz, 1H), 7.05 (d, J=9.20 Hz, 1H), 6.98 (d, J=8.80 Hz, 2H), 5.74-5.48 (m, 3H), 3.87 (s, 3H), 3.76 (s, 3H), 2.48 (s, 3H), 1.67 (d, J=6.40 Hz, 3H)

Step O: To a solution of methyl (R)-6-chloro-3-((1-(2-cyano-3-((4-methoxybenzyl) oxy)-7-methylquinoxalin-5-yl) ethyl) amino) picolinate (1.30 g, 2.51 mmol, 1.00 eq.) in dichloromethane (10.0 mL) was added trifluoroacetic acid (4.61 g, 40.4 mmol, 3.00 mL, 16.1 eq.) at 0° C. The mixture was stirred at 25° C. for 1 hour. After completion, the mixture was concentrated to give methyl (R)-6-chloro-3-((1-(2-cyano-3-hydroxy-7-methylquinoxalin-5-yl) ethyl) amino) picolinate (2.50 g crude) as a yellow solid. LCMS [M+1] ⁺=398.2.

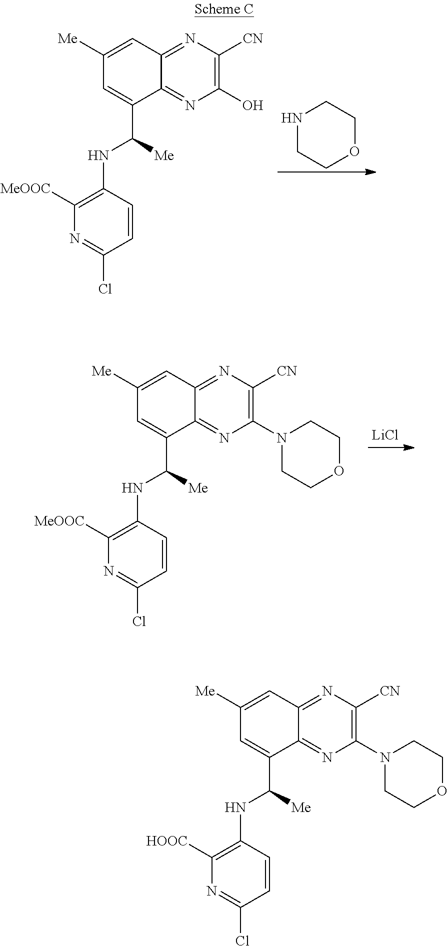

Step P: To a solution of methyl (R)-6-chloro-3-((1-(2-cyano-3-hydroxy-7-methylquinoxalin-5-yl) ethyl) amino) picolinate (50.0 mg, 113 μmol, 1.00 eq.) and morpholine (14.8 mg, 170 μmol, 14.9 μL, 1.50 eq.) in DMF (0.50 mL) was added PyBOP (88.3 mg, 170 μmol, 1.50 eq.) and N,N-diisopropylethylamine (43.9 mg, 339 μmol, 59.1 μL, 3.00 eq.), and the mixture was stirred at 25° C. for 1 hour. After this time, water (1.00 mL) was slowly added to the mixture and a yellow precipitate was formed. The suspension was then filtered, and the cake was collected and dried under vacuum to give methyl (R)-6-chloro-3-((1-(2-cyano-7-methyl-3-morpholinoquinoxalin-5-yl) ethyl) amino) picolinate (52.0 mg, 111 μmol, 98.5% yield) as a yellow solid. LCMS [M+1]⁺=467.2.

Step Q: To a solution of methyl (R)-6-chloro-3-((1-(2-cyano-7-methyl-3-morpholinoquinoxalin-5-yl) ethyl) amino) picolinate (52.0 mg, 111 μmol, 1.00 eq.) in DMSO (0.50 mL) was added lithium chloride (47.2 mg, 1.11 mmol, 22.8 μL, 10.0 eq.), and the mixture was stirred at 120° C. for 6 hours. The mixture was then cooled to 25° C., filtered to give a filtrate, and the filtrate was purified by prep-HPLC (column: YMC-Actus Triart C18 150×30 mm×7 μm; mobile phase: phase A: 0.225% formic acid in water, phase B: acetonitrile; gradient: 55%-85% B) to give (R)-6-chloro-3-((1-(2-cyano-7-methyl-3-morpholinoquinoxalin-5-yl) ethyl) amino) picolinic acid (8.93mg, 19.3 μmol, 17.4% yield, 98.1% purity) as a white solid. LCMS [M+1] ⁺=453.2 .

¹H NMR (400 MHZ, CD₃OD) δ=7.66 (d, J=1.6 Hz, 1H), 7.63 (s, 1H), 7.19 (d, J=9.2 Hz, 1H), 7.02 (d, J=9.2 Hz, 1H), 5.53 (q, J=6.8 Hz, 1H), 3.98-3.86 (m, 4H), 3.83-3.72 (m, 4H), 2.46(s, 3H), 1.73 (d, J=6.8 Hz, 3H).

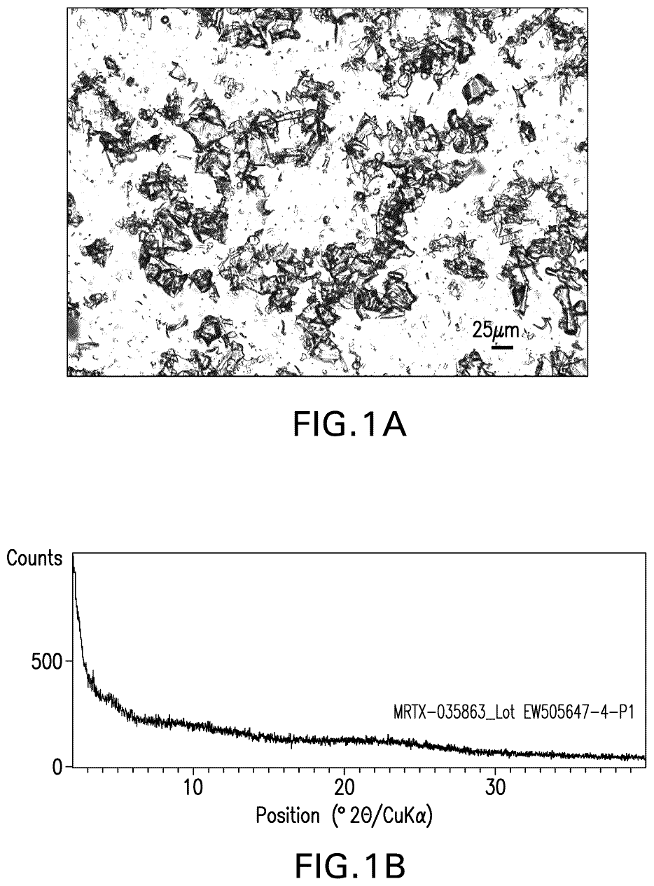

A polarized light microscopy (PLM) image and PXRD of the amorphous form produced in this example are shown in FIGS. 1A and 1B, respectively.

### Example 2. Cell Assays & Cell Viability of Compound 1

This Example illustrates that representative compounds of the invention inhibit the formation of phospho-AKT (pAKT) in a cell and decrease the viability of cells.

The ability of a Compound 1 to inhibit the formation of pAKT was measured using alphaLISA Surefire Ultra AKT 1/2/3 (pS473) Assay Kit (#ALSU-PAKT-B50K) was obtained from Perkin Elmer (Waltham, MA). The ability of Compound 1 to decrease the viability of cells was measured using CellTiter-Glo 2.0 (CTG) Luminescent Cell Viability Assay (#G9241) obtained from Promega (Madison, WI).

To prepare assay plates for pAKT alphaLISA assays, cells were trypsinized, resuspended in fresh media, and viable cells were counted utilizing trypan blue exclusion. Prior to seeding, cells were washed with PBS and resuspended in HBSS (Gibco, #14025092). T47D (12,000/w), SKBR3(12,000/w), or MKN1 (24,000/w) cells were seeded at 12 μl per well in a solid white flat bottom 384 cell culture plate (Perkin Elmer #6007680).

Immediately after seeding, cells were dosed using an Echo Liquid Handler (Beckman Coulter) with compounds at a 10.4 μm starting concentration and serially diluted (1:4) for a total of 10concentrations. 14 vehicle (DMSO) and 14 positive control (Alpelisib @ 3.125 μm) wells were included on each assay plate. Cells were incubated with the compounds (solubilized in DMSO) for approximately 24 hours at 37° C. After 24 hours of treatment, cells were lysed with 3 μl of the 5×lysis buffer (provided and incubated at room temperature for 15 min on a microtiter plate shaker). Once cells were sufficiently lysed, 7.5 μl of the acceptor bead mix (made using the manufacturers recommended dilutions) was added to each well and left on the microtiter plate shaker for 1 min prior to incubating plates at room temperature for 1 hour, in the absence of light. After 1 hour of incubation with the acceptor bead mix, 7.5 μl of the donor bead mix (made using the manufacturers recommended dilutions) was added to each well and left on the microtiter plate shaker for 1 min prior to incubating plates at room temperature overnight in the absence of light. Plates were then imaged the following day using a CLARIOstar microplate reader (BMG Labtech, Germany).

Percent of control values were calculated by subtracting the average signal from the positive control (alpelisib) treated wells from all treated wells (including DMSO control wells) and then divided by the average signal from the vehicle DMSO treated control wells. Percent of vehicle control values were plotted as log (inhibitor) vs. response-Variable slope (four parameters) for curve fitting and IC50 values were determined using XLfit.

To prepare assay plates for viability assays, cells were trypsinized, resuspended in fresh media, and viable cells were counted utilizing trypan blue exclusion. T47D or SKBR3 cells were seeded at 1000 cells in 30 μl per well in a solid white flat bottom 384 cell culture plate (Perkin Elmer #6007680) and incubated at 37° C. overnight.

Assay day 1, cells were dosed using an Echo Liquid Handler (Beckman Coulter) with compounds at a 10 μm starting concentration and serially diluted (1:4) for a total of 10concentrations. Cells were incubated for approximately 72 hours with the compounds (solubilized in DMSO) at 37° C. After 72 hours of treatment, cell plates were equilibrated to room temperature before adding 15 μl of CTG to each well, plates were then covered in aluminum foil to protect from light, incubated at room temperature for 30 minutes on a microtiter plate shaker, and luminescence readings were collected using a CLARIOstar microplate reader (BMG Labtech, Germany). Percent of vehicle control values were plotted as log (inhibitor) vs. response-Variable slope (four parameters) for curve fitting and IC50 values were determined using XLfit.

Table 1 reports the results of these cell studies.

TABLE 1

| | T47D (H1047R) | T47D (H1047R | SKBR3 (WT) |
|---|---|---|---|
| | pAKT 24 | mutant) Viability | Viability |
| | hr (IC₅₀) | IC₅₀ (nM) | IC₅₀ (nM) |
| Compound 1 | 1.8 | 24.0 | 3502 |

### Example 3: Formation of Crystalline Form of Compound 1

The amorphous solids of Compound 1 as prepared in Example 1 was measured in 20 single solvents at room temperature and crystallization screening was then conducted in the same set of solvents. For each solvent tested, a slurry of the amorphous solids and the solvent was prepared with a solvent volume of 200 mg/mL and the amorphous solids were allowed to dissolve. The slurry was then agitated with a stir bar for approximately 1 hour at room temperature. The wet solids were then analyzed by powder X-ray diffraction (PXRD). The solids were collected and dried under vacuum for 1 day and the crystalline form was confirmed by PXRD. Only one crystalline form was obtained and designated as Form 1. The results of the solubility of the amorphous solids of Compound 1 and crystallizing screening are shown in Table 2.

TABLE 2

| | | | | | Solvent | | | | |
|---|---|---|---|---|---|---|---|---|---|
| | Solids | | Solvent | b.p | added | Solubility | Crystallization screening | | |
| No. | (mg) | Solvents | Class | (° C.) | (mL) | (mg/mL) | t₀ | 1 d | PXRD |
| 1 | 5.26 | MeOH | 2 | 64.7 | 0.025 | >210.4 | MS | HS | Form 1 |
| 2 | 5.37 | EtOH | 3 | 78.37 | 0.125 | 43.0-53.7 | MS | HS | Form 1 |
| 3 | 5.04 | IPA | 3 | 82.5 | 0.35 | 14.4-16.8 | MS | HS | Form 1 |
| 4 | 5.52 | IPAc | 3 | 89 | 0.025 | >220.8 | MS | HS | Form 1 |
| 5 | 5.10 | EtOAc | 3 | 77.1 | 0.025 | >204.0 | MS | HS | Form 1 |
| 6 | 5.89 | nPA | 3 | 97 | 0.125 | 47.1-58.9 | MS | HS | Form 1 |
| 7 | 5.06 | ACN | 2 | 82 | 0.025 | >202.4 | MS | HS | Form 1 |
| 8 | 4.77 | MEK | 3 | 79.64 | 0.025 | >190.8 | MS | HS | Form 1 |
| 9 | 4.72 | MIBK | 2 | 117-118 | 0.025 | >188.8 | MS | HS | Form 1 |
| 10 | 5.12 | MTBE | 3 | 55.2 | 0.05 | 102.4-220.8 | MS | HS | Form 1 |
| 11 | 5.37 | THF | 2 | 66 | 0.025 | >214.8 | CL | CL | — |
| 12 | 5.52 | 1,4- | 2 | 101 | 0.025 | >220.8 | CL | CL | — |
| | | Dioxane | | | | | | | |
| 13 | 5.75 | 2-MeTHF | 2 | 80.2 | 0.025 | >230.0 | MS | HS | Form 1 |
| 14 | 5.14 | heptane | 3 | 98.42 | 4 | <1.3 | MS | HS | Form 1 |
| 15 | 5.11 | toluene | 2 | 110.6 | 0.025 | >204.4 | MS | HS | Form 1 |
| 16 | 5.25 | water | — | 100 | 4 | <1.3 | MS | HS | Amorphous |
| 17 | 5.75 | acetone | 3 | 56 | 0.025 | >230.0 | MS | HS | Form 1 |
| 18 | 4.80 | DCM | 2 | 39.6 | 0.025 | >192.0 | CL | CL | — |
| 19 | 5.40 | DMF | 2 | 153 | 0.025 | >216.0 | CL | CL | — |
| 20 | 6.02 | DMSO | 3 | 189 | 0.025 | >240.8 | CL | CL | — |

TS: thin slurry;
MS: medium slurry;
HS: heavy slurry,
CL: clear:
GR: grease

### Example 4: Characterization of Crystalline Form of Compound 1

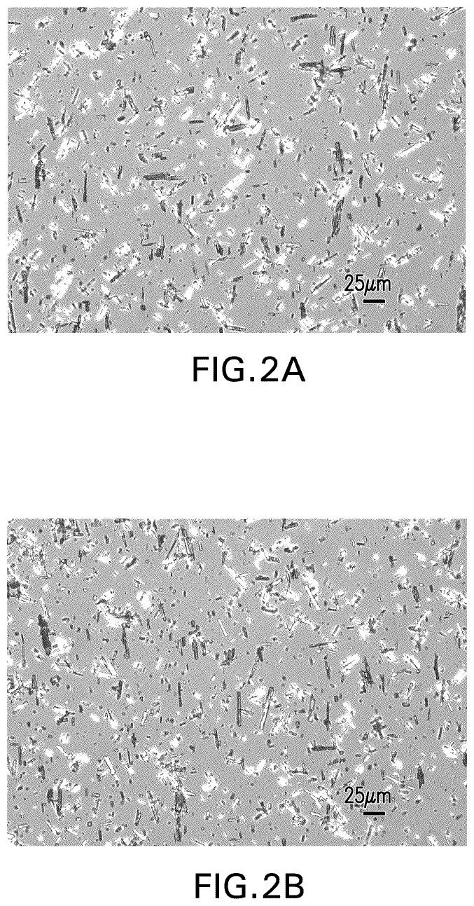

The crystalline form of Compound 1 is highly crystalline and has a needle/thin rod-like morphology. FIG. 2A and 2B are polarized light microscopy (PLM) images of this crystalline form of Compound 1. Additionally, the crystalline form of Compound 1 was characterized by PXRD, DSC, TGA, ¹H-NMR, HPLC, and DVS. The solvent used for the ¹H-NMR was methanol. The results are summarized in Table 3 and shown in FIGS. 2-6.

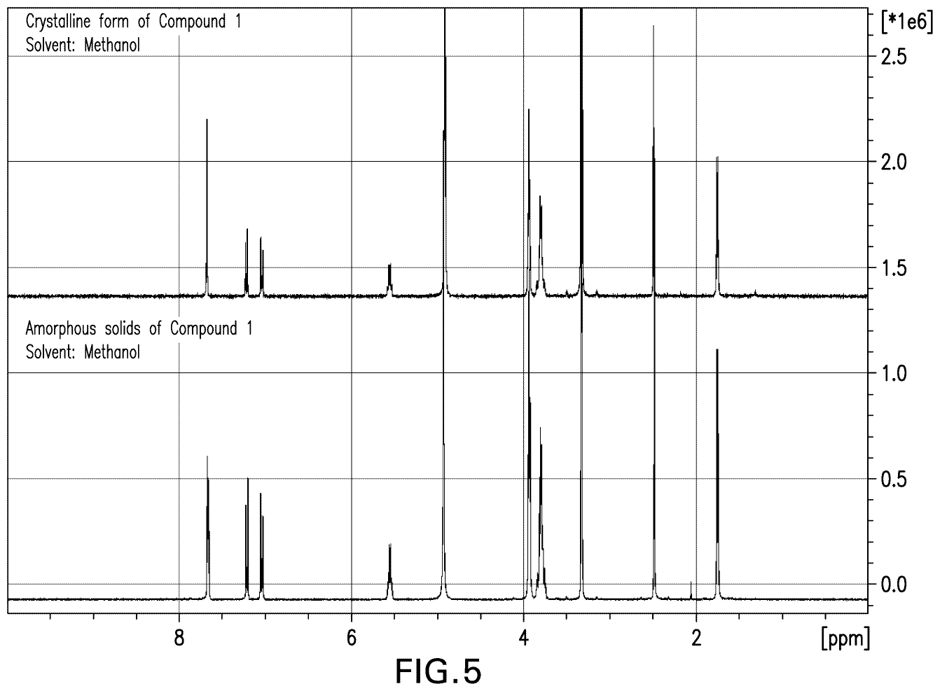

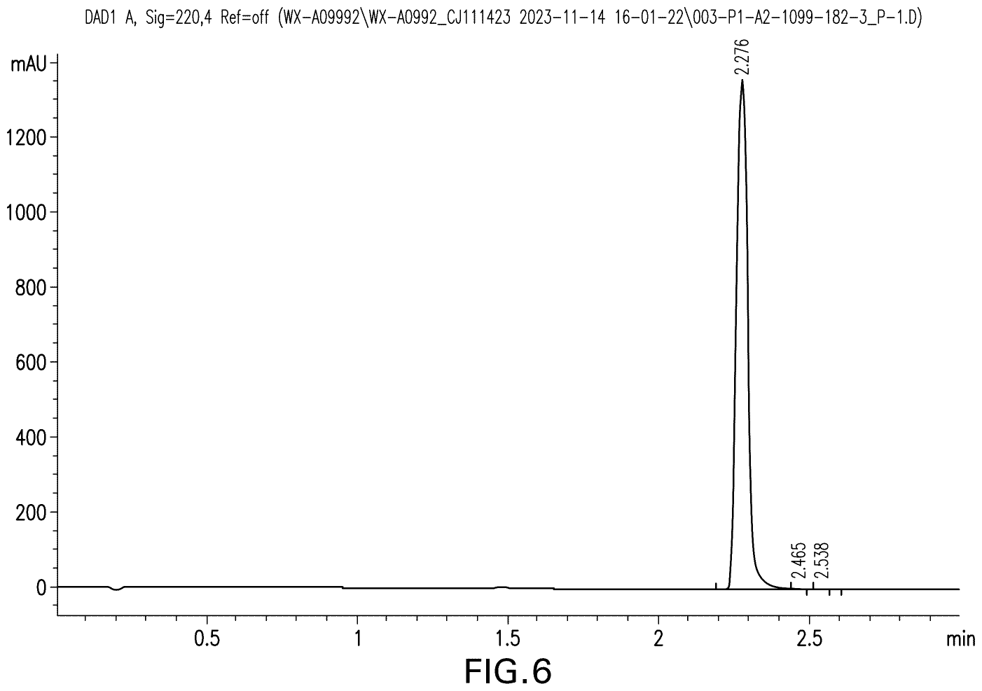

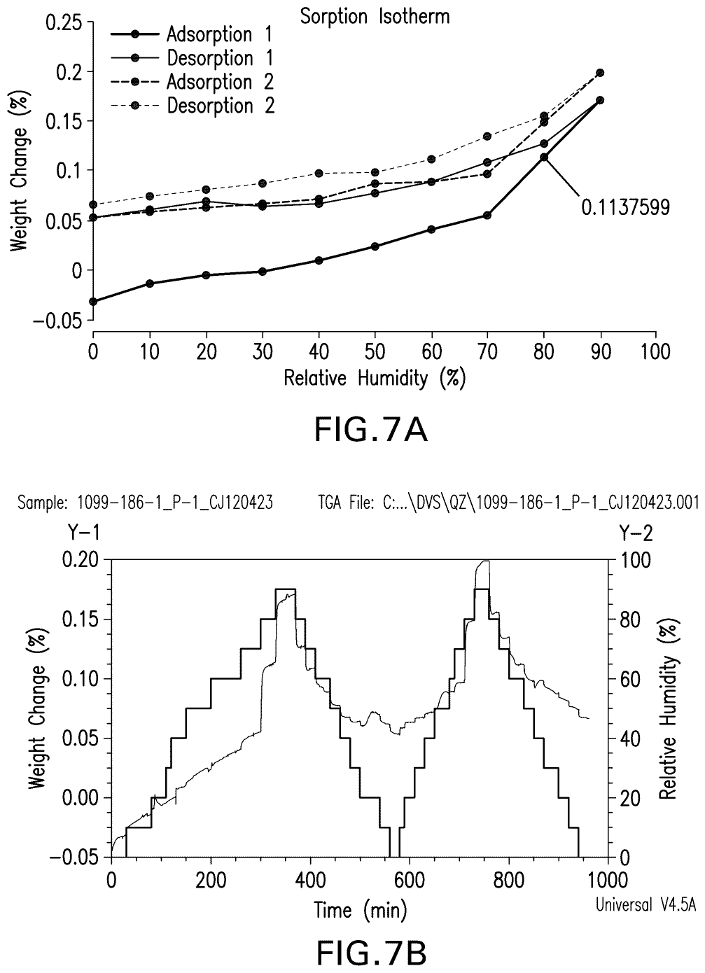

TABLE 3

| Technique | Data | Results |
|---|---|---|
| PXRD | FIG. 3 | Highly crystalline |
| DSC/TGA | FIG. 4 | Endotherm at 177.61° C. (peak temp) |
| | | 0.041% weight loss observed before 100° C. |
| ¹H-NMR | FIG. 5 | Consistent with chemical structure |
| HPLC | FIG. 6 | Single peak, 99.9 Area % |
| DVS | FIG. 7A | weight gain at 80% RH: 0.11%, non-hygroscopic |
| | & 7B | |

Table 4 described the XRD pattern of the crystalline form of Compound 1 shown in FIG. 3.

TABLE 4

| Pos. (°2θ) | Intensity | Height (cts) |
|---|---|---|
| 7.56961 | Medium | 14218.4 |
| 11.12275 | High | 18843.9 |
| 13.15453 | Medium | 8632.4 |
| 13.98141 | Medium | 16809.3 |
| 18.86539 | High | 13264.2 |
| 19.77346 | Medium | 8885.5 |
| 22.27129 | Medium | 13735.9 |
| 23.03792 | Medium | 16987.3 |
| 23.62856 | Medium | 19036 |
| 24.09841 | Medium | 25176.3 |
| 24.34751 | High | 23205.4 |

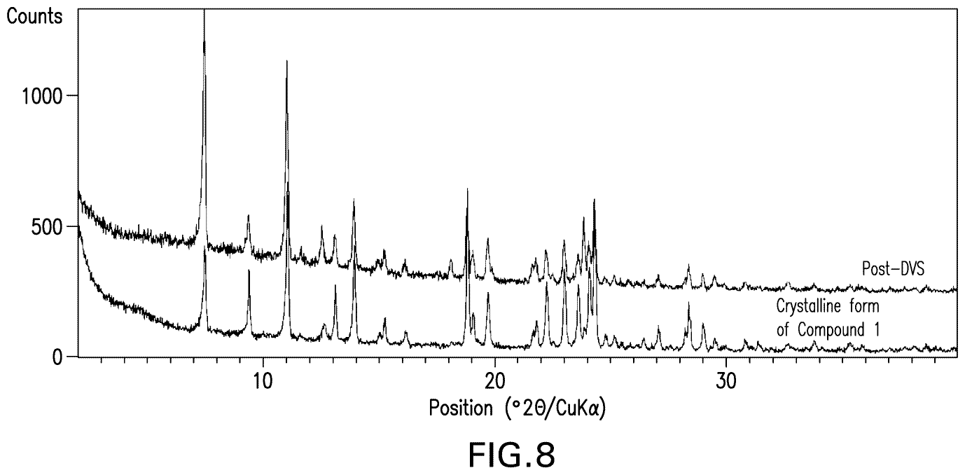

The crystalline form has a high melting temperature (˜177-180° C.) with no thermal degradation until >210° C. The crystalline form appears to be an anhydrous form and is non-hygroscopic. The crystalline form was analyzed by PXRD before and after DVS. The PXRD from before and after DVS are shown in FIG. 8 and showed no form change.

### Example 5: Unit Cell Determination of Crystalline Form of Compound 1

The single crystal X-ray diffraction studies were carried out on a Bruker ApexII-Ultra CCD diffractometer equipped with Mo Kα radiation (λ=0.7107 Å).

A 0.16×0.16×0.11 mm piece of a crystal of Compound 1 was mounted on a Cryoloop with Paratone oil. Data were collected in a nitrogen gas stream at 100(2) K using ϕ and ω scans. Crystal-to-detector distance was 40 mm and exposure time was 10 seconds depending on the 20range per frame using a scan width of 0.70°. Data collection was 99.7% complete to 25.242° in θ. A total of 26277 reflections were collected covering the indices, −8<=h<=9, −23<=k<=16,−37<=1<=24. 8630 reflections were found to be symmetry independent, with a Rᵢₙₜ of 0.0564.Indexing and unit cell refinement indicated a Primitive, Orthorhombic lattice. The space group was found to be P212121. The data were integrated using the Bruker SAINT Software program and scaled using the SADABS software program. Solution by direct methods (SHELXT) produced a complete phasing model consistent with the proposed structure.

All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. Crystallographic data are summarized in Table 5.

TABLE 5

| Empirical formula | C22 H21 Cl N6 O3 | |
|---|---|---|
| Formula weight | 452.90 | |
| Temperature | 100.15 | K |
| Wavelength | 0.71073 | Å |
| Crystal system | Orthorhombic | |
| Space group | P2₁2₁2₁ | |
| Unit cell dimensions | a = 7.4125(7) Å, α = 90° | |
| | b = 18.7909(17) Å, β = 90° | |
| | c = 30.418(3) Å, γ = 90° | |
| Volume | 4236.8(7) | Å³ |
| Z | 8 | |
| Density (calculated) | 1.420 | Mg/m³ |
| Absorption coefficient | 0.219 | mm⁻¹ |
| F(000) | 1888 | |
| Crystal size | 0.16 × 0.16 × 0.11 | mm³ |

| | |
|---|---|
| Theta range for data collection | 1.274 to 26.391°. |
| Index ranges | −8 <= h <= 9, −23 <= k <= |
| | 16, −37 <= l <= 24 |
| Reflections collected | 26277 |
| Independent reflections | 8630 [R(int) = 0.0564] |
| Completeness to theta = 25.242° | 99.7% |
| Max. and min. transmission | 0.4908 and 0.3529 |
| Refinement method | Full-matrix least-squares on F² |
| Data/restraints/parameters | 8630/0/583 |
| Goodness-of-fit on F² | 1.044 |
| Final R indices [I > 2sigma(I)] | R1 = 0.0534, wR2 = 0.1165 |
| R indices (all data) | R1 = 0.0665, wR2 = 0.1226 |
| Absolute structure parameter | 0.03(3) |
| Largest diff. peak and hole | 0.569 and −0.480 e · Å⁻³ |

### Example 6: Quantitative NMR Crystalline Form of Compound 1

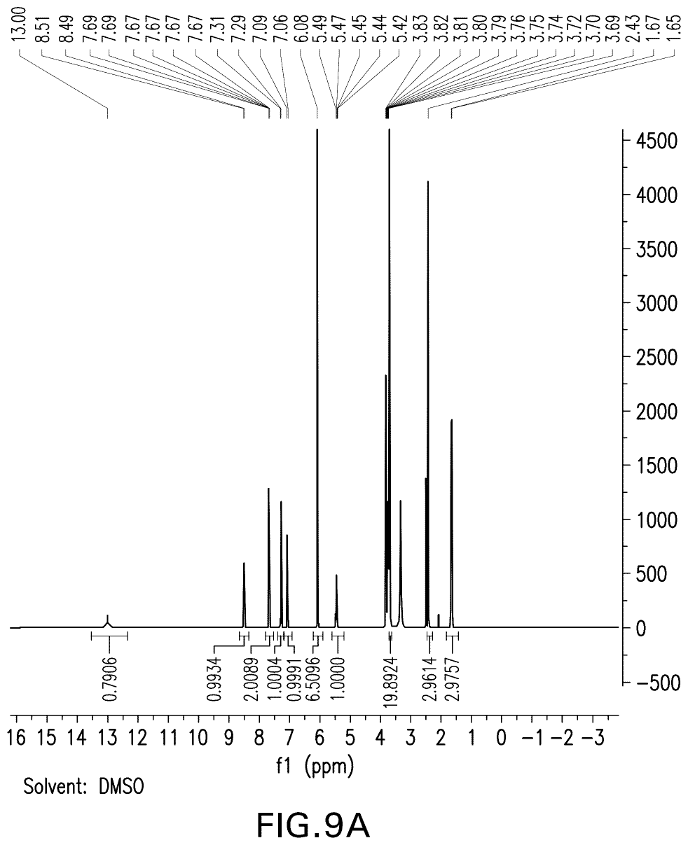

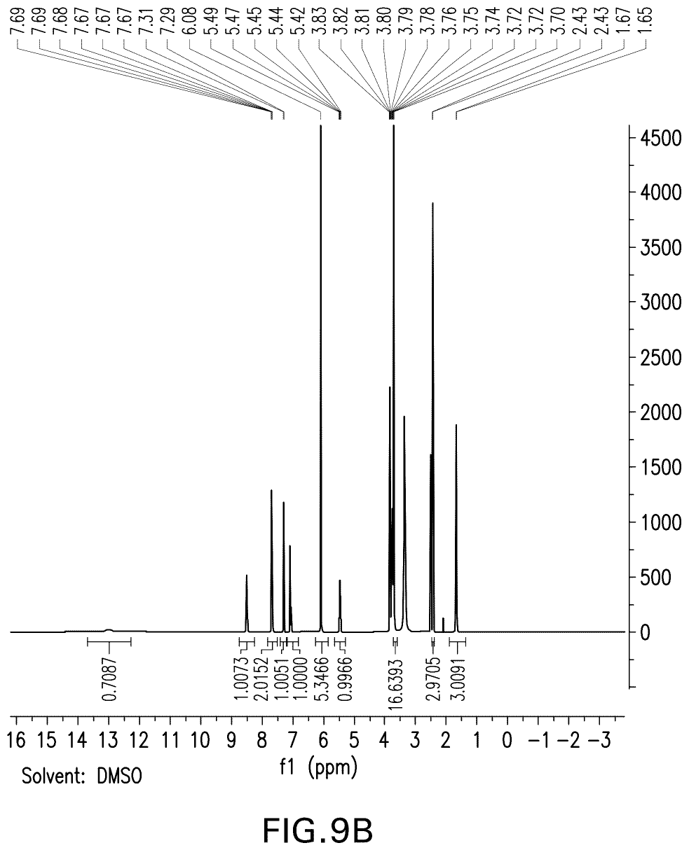

Quantitative NMR (Q-NMR) analysis was performed on the amorphous solids of Compound 1 and crystalline solids of Compound 1 to determine purity/potency of the two materials. The solvent used for the Q-NMR measurement was DMSO. Compared to 96.07 wt % of the amorphous material, the crystalline form showed an upgraded purity of 98.50 wt %. The Q-NMR results of two samples of the crystalline form of Compound 1 are shown in Table 6 and FIGS. 9A and 9B.

TABLE 6
formula: Pa = (ms/mA)(MA/Ms) (IA/NA) (Ns/Is) Ps

| | W spl, mg | Wstd, mg | M spl | M std | A spl | A std | n spl | n std | |
|---|---|---|---|---|---|---|---|---|---|
| Sample No. | wt, mg | wt, mg | analyte | standard | analyte | Standard | Na | Ns | P std, % |
| 1 | 11.60 | 9.25 | 452.90 | 168.19 | 11.9389 | 6.5096 | 12 | 3 | 99.99 |
| 2 | 9.74 | 6.35 | 452.90 | 168.19 | 12.0068 | 5.3466 | 12 | 3 | 99.99 |

While the invention has been described in connection with specific embodiments thereof, it will be understood that it is capable of further modifications and this application is intended to cover any variations, uses, or adaptations of the invention following, in general, the principles of the invention and including such departures from this disclosure as come within known or customary practice within the art to which the invention pertains and as may be applied to the essential features hereinbefore set forth, and as follows in the scope of the appended claims.

We claim:

1. A crystalline form of (R)-6-chloro-3-((1-(2-cyano-7-methyl-3-morpholinoquinoxalin-5-yl) ethyl) amino) picolinic acid having a powder X-ray diffraction (PXRD) pattern comprising a peak at a two-theta angle of 11.1°±0.2°.

2. The crystalline form of claim 1, wherein the crystalline form has a powder X-ray diffraction (PXRD) pattern comprising a peak at a two-theta angle of 18.8°±0.2°.

3. The crystalline form of claim 1, wherein the crystalline form has a powder X-ray diffraction (PXRD) pattern comprising a peak at a two-theta angle of 24.3°±0.2°.

4. The crystalline form of claim 1, wherein the crystalline form has a powder X-ray diffraction (PXRD) pattern comprising a peak at a two-theta angle of 7.6°±0.2°.

5. The crystalline form of claim 1, wherein the crystalline form has a powder X-ray diffraction (PXRD) pattern comprising a peak at a two-theta angle of 13.1°±0.2°.

6. The crystalline form of claim 1, wherein the crystalline form has a powder X-ray diffraction (PXRD) pattern comprising a peak at a two-theta angle of 14.0°±0.2°.

7. The crystalline form of claim 1, wherein the crystalline form has a powder X-ray diffraction (PXRD) pattern comprising a peak at a two-theta angle of 19.7°±0.2°.

8. The crystalline form of claim 1, wherein the crystalline form has a powder X-ray diffraction (PXRD) pattern comprising a peak at a two-theta angle of 22.2°±0.2°.

9. The crystalline form of claim 1, wherein the crystalline form has a powder X-ray diffraction (PXRD) pattern comprising a peak at a two-theta angle of 23.0°±0.2°.

10. The crystalline form of claim 1, wherein the crystalline form has a powder X-ray diffraction (PXRD) pattern comprising a peak at a two-theta angle of 23.6°±0.2°.

11. The crystalline form of claim 1, wherein the crystalline form has a powder X-ray diffraction (PXRD) pattern comprising a peak at a two-theta angle of 24.1°±0.2°.

12. The crystalline form of claim 1, wherein the crystalline form has a powder X-ray diffraction (PXRD) pattern comprising peaks at a two-theta angle of 11.1°±0.2°, 18.8°±0.2°,and 24.3°±0.2°.

13. The crystalline form of claim 1, wherein the crystalline form has a powder X-ray diffraction (PXRD) pattern comprising peaks at a two-theta angle of 11.1°±0.2°, 18.8°±0.2°, 24.3°±0.2°, 7.6°±0.2°, 13.1°±0.2°, 14.0°±0.2°, 19.7°±0.2°, 22.2°±0.2°, 23.0°±0.2°, 23.6°±0.2°, and 24.1°±0.2°.

14. A crystalline form of (R)-6-chloro-3-((1-(2-cyano-7-methyl-3-morpholinoquinoxalin-5-yl) ethyl) amino) picolinic acid having a powder X-ray diffraction (PXRD) pattern a powder X-ray diffraction (PXRD) pattern substantially shown in FIG. 3.

15. A crystalline form of (R)-6-chloro-3-((1-(2-cyano-7-methyl-3-morpholinoquinoxalin-5-yl) ethyl) amino) picolinic acid having a unit cell of a =7.4±0.2 Å, b=18.8±0.2 Å, c±30.4 ±0.2 Å, α±90°, ß±90°, and γ±90°.

16. A crystalline form of (R)-6-chloro-3-((1-(2-cyano-7-methyl-3-morpholinoquinoxalin-5-yl) ethyl) amino) picolinic acid having an endothermic differential scanning calorimetric (DSC) peak temperature within ±2% of 177° C.

17. The crystalline form of claim 16, wherein the endothermic differential scanning calorimetric (DSC) peak temperature is within ±1% of 177° C.

18. The crystalline form of claim 16, wherein the endothermic differential scanning calorimetric (DSC) peak temperature is within ±0.5% of 177° C.

19. The crystalline form of any of claim 1, wherein the crystalline form is anhydrous.

20. The crystalline form of any of claim 1, wherein the crystalline form is non-hygroscopic.

21. The crystalline form of any of claim 1, wherein the crystalline form has a thermal gravimetric analysis (TGA) plot comprising a mass loss of about 0.041% when heated from about 25° C. to about 100° C.

22. The crystalline form of any of claim 1, wherein the crystalline form has substantially no thermal degradation at temperatures below about 210° C.

23. The crystalline form of any of claim 1, wherein the crystalline form has a purity of at least 97% by weight of (R)-6-chloro-3-((1-(2-cyano-7-methyl-3-morpholinoquinoxalin-5-yl) ethyl) amino) picolinic acid.

24. A pharmaceutical composition comprising the crystalline form of any of claim 1 and a pharmaceutically acceptable carrier.

25. A method of treating a disease or disorder associated with modulation of phosphoinositide 3-kinase (PI3K), comprising administering to a patient in need thereof a therapeutically effective amount of a crystalline form of any one of claim 1.

26. The method of any one of claim 25, wherein the disease or disorder is a cancer.
